(19) United States
(12) Plant Patent Application Publication
(10) Pub. No.: US 2008/0184414 P1
(43) Pub. Date: Jul. 31, 2008

(54) Lantana plant named 'Bante Pinka07'

(76) Inventor: Shifeng Pan, Hollister, CA (US)

Correspondence Address: JONDLE & ASSOCIATES P.C., 858 HAPPY CANYON ROAD SUITE 230, CASTLE ROCK, CO, 80108, US

(21) Appl. No.: 11/654,870

(22) Filed: Jan. 17, 2007

(51) Int. Cl. A01H 5/00

(52) U.S. Cl. PLT/227

(57) ABSTRACT

A lantana plant particularly distinguished by lavender-pink flowers, a continuous, abundant and mid-season flowering response, compact growth, low mounding and good branching ability, is disclosed.

### GENUS AND SPECIES

Lantana camara

### VARIETY DENOMINATION

‘Bante Pinka07’

### BACKGROUND OF THE NEW PLANT

The present invention comprises a new and distinct cultivar of Lantana, botanically known as Lantana camara and hereinafter referred to by the cultivar name ‘Bante Pinka07’. The new cultivar originated from a pollination made in July 2003 in Gilroy, Calif. The new cultivar was created from the crossing of ‘22-1’, a fuchsia female (unpatented) proprietary line with ‘F13A-1’, a pink male (unpatented) proprietary line.

The new cultivar was created in July of 2003 in Gilroy, Calif. and has been asexually reproduced repeatedly by vegetative cuttings and tissue culture in Gilroy, Calif. and Andijk, The Netherlands over a two and one-half-year period. The plant has also been trialed at Gilroy, Calif., Andijk, The Netherlands and Hillscheid, Germany. The present invention has been found to retain its distinctive characteristics through successive asexual propagations.

Plant Breeder's Rights for this cultivar were applied for in Germany on Nov. 27, 2006.

### SUMMARY OF THE INVENTION

The following are the most outstanding and distinguishing characteristics of the new cultivar when grown under normal horticultural practices in Gilroy, Calif.
1. Lavender-pink flowers;
2. A continuous, abundant and mid-season flowering response;
3. A compact and low mounding plant habit; and
4. Good branching ability.

### DESCRIPTION OF THE PHOTOGRAPHS

This new lantana plant is illustrated by the accompanying photographs which show overall plant habit including blooms, buds and foliage of the plant; the colors shown are as true as can be reasonably obtained by conventional photographic procedures. The photos are of 15-16 week-old plants in a greenhouse in Hillschied, Germany in September of 2006.

FIG. 1 shows the overall plant habit, including inflorescences, buds and foliage of the whole plant

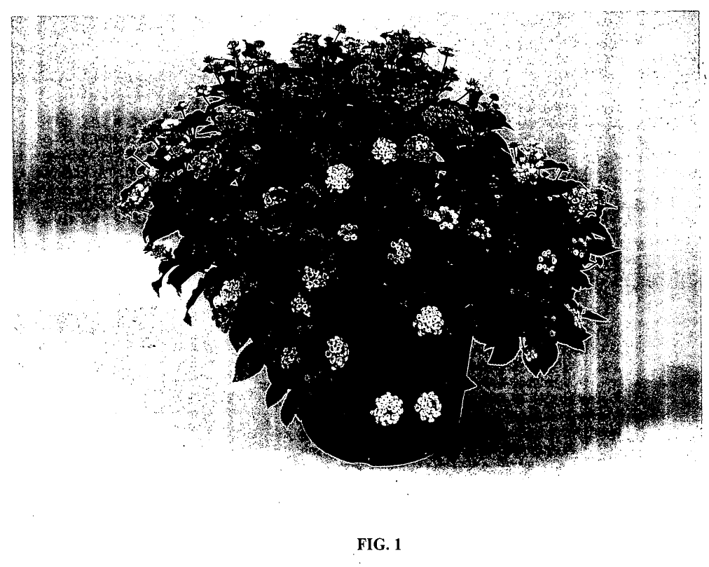

FIG. 2 shows a close-up of the mature inflorescence.

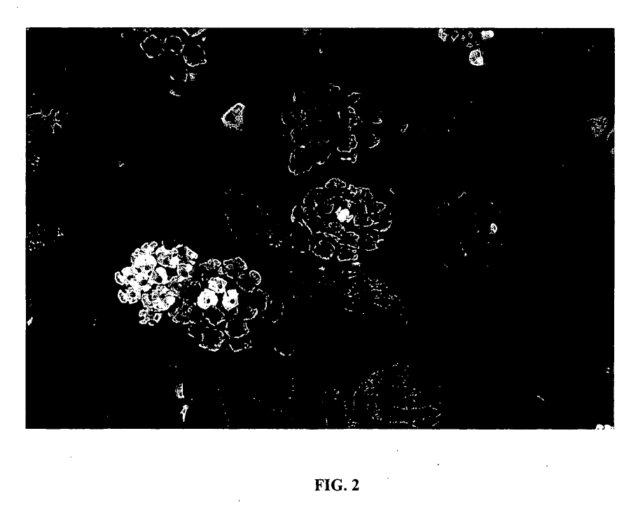

### DESCRIPTION OF THE NEW CULTIVAR

The following detailed descriptions set forth the distinctive characteristics of ‘Bante Pinka07’. The data which define these characteristics were collected from asexual reproductions carried out in Gilroy, Calif. The plant history was taken on 26-week-old plants, grown in Gilroy, Calif. in 6″ pots. Observations were made in summer and early fall. The plants had one plant growth regulator treatment and one terminal pinch after cuttings were transplanted to the 4″ pots then transferred to 6″ inch pots. Color readings were taken under natural light. Color references are primarily to the RHS Colour Chart of The Royal Horticultural Society of London (RHS) (2001 edition).

### DETAILED BOTANICAL DESCRIPTION

Classification:
   Family.—Verbenaceae.
   Botanical.—Lantana camara.
   Common name.—Lantana.
Parentage:
   Female.—‘22-1’, a fuschia proprietary line (unpatented).
   Male.—‘F13A-1’ a pink proprietary line (unpatented).
Growth:
   Form.—Semi-woody, herbaceous; grown as an annual or perennial.
   Growth and branching habit.—Compact, low mounding, good branching, pinching of the apices enhances the number of branches and results in a tighter plant habit.
   Height (measured from the top of the soil).—15.0 cm to 20.0 cm.
   Width (horizontal plant diameter).—30.0 cm to 35.0 cm.
   Time to produce a finished flowering plant.—12 to 13 weeks for a 12-cm pot in the spring.
   Outdoor plant performance.—Use in containers and hanging baskets.
   Time to initiate and develop roots.—Approximately 24 days.
   Root description.—Fibrous, fleshy, white, self-branching.
Stems:
   Number of branches per plant.—6 to 8.
   Length.—12.0 cm to 15.0 cm.
   Diameter (measured from the midpoint).—0.20 cm to 0.25 cm.
   Internode length.—1.3 cm to 3.0 cm.
   Color.—Immature (growing) stems: RHS 144A Mature (woody) stems: RHS 165A with RHS 159A stripes.
   Anthocyanin.—Absent.
   Texture.—Glandular hairs; Scabrous, some hairs with a black ring around their base.
Leaves:
   Arrangement.—Opposite.
   Fragrance.—Somewhat spicey.
   Immature leaf.—Color: Upper surface: Closest to RHS 137A Lower surface: Closest to RHS 137C.
   Mature leaf.—Color: Upper surface: Closest to RHS 139A, but darker Lower surface: RHS 146B Length: 5.9 cm to 6.2 cm Width: 3.5 cm to 4.2 cm Shape: Oblanceolate to elliptical Apex: Acute Base: Attenuate Margin: Serrate Texture: Rugose; glandular hairs; Scabrous with bristles in the mid-vein having a black ring around the base. Venation: Pinnate Venation color: RHS 144B for the lower surface, the upper surface is indistinct.
   Petioles.—Length: 0.8 cm to 1.4 cm Diameter: 0.15 cm Color: RHS 144B Texture: Glandular hairs; scabrous, some bristles with black rings around the base.
Flower buds:
   Shape.—Tubular.
   Length.—0.9 cm to 1.0 cm.
   Diameter (at the tip).—0.2 cm.
   Color (at tight bud).—RHS 198A.
Inflorescence:
   Blooming habit.—Flowers continuously from Spring through Fall; Inflorescences usually appear in pairs from a nodium with one inflorescence per leaf.
   Fragrance.—Somewhat spicey.
   Inflorescence type.—Umbel-like flattened semi-sphere, florets are sessile on an ovate receptacle.
   Horizontal diameter of inflorescence.—3.3 cm to 3.5 cm.
   Vertical depth of inflorescence.—2.0 cm to 2.2 cm.
   Number of inflorescences per plant.—20 to 30.
   Number of florets per inflorescence.—27 to 31.
   Peduncle.—Length: 2.5 cm to 4.0 cm Diameter: 0.1 cm to 0.15 cm Color: RHS 144A to RHS 144B Texture: Glandular hairs; Scabrous.
   Pedicels.—None, flowers are sessile.
Flowers:
   Floret form.—Salver-shaped to shallow cup-shaped and zygomorphic, with a relatively long tube at the base. Each flower is subtended by a single bract.
   Corolla diameter.—1.0 cm to 1.2 cm.
   Floret depth.—2.0 cm.
   General color.—Mostly light-medium yellow in the very opening stages, then turning through varying hues of light lavender-pink and lavender-pink mature florets. Color intensities can vary with cultural growing conditions.
   Immature flower (face).—Color: Upper surface: RHS 9B at the corolla opening; RHS 9D towards the margins; changing to RHS 55D to RHS 55B, with a little RHS 23C at the corolla opening. Lower surface: RHS 8D.
   Mature flower (face).—Color: Upper surface: Between RHS 55D and RHS 55C; maturing to RHS N57D Lower surface: RHS 56C Corolla tube: Color: Inner surface: RHS 27B Outer surface: RHS 36B Texture: Densely pubescent Length: 1.0 cm Diameter (at opening): 0.25 cm.
   Petals.—Length of lobe: 0.5 cm to 0.6 cm Diameter of lobe: 0.5 cm to 0.6 cm Lobe shape: Obovate Apex: Rounded Base: Fused Margin: Entire Degree of lobation: Moderate Texture: Papillose, with a few hairs around the opening.
   Bract.—Color: RHS 144A, but darker Length: 0.4 cm Diameter: 0.15 cm Shape: Short, lanceolate Apex: Acute Margin: Entire Base: Fused Texture: Glandular hairs, densely pubescent.
   Calyx.—General: Composed of 5 fused sepals forming a short tube around the base of each floret Shape: Tube-shaped with two short lobes Length: 0.2 cm Diameter: 0.1 cm Color: Upper surface: RHS 44C Apex (of calyx lobe): Rounded Base (of sepals): Fused Margin (of sepals): Side-margins are fused, but tips are shallowly fringed. Texture: Densely pubescent.
Reproductive organs:
   Stamens.—Quantity: 4 Filament color: RHS 27B Filament diameter: 0.5 cm to 0.7 cm Filament length: 0.05 cm Anther color: RHS 13C Anther length: 0.5 cm to 0.75 cm Anther shape: Oval Pollen amount: Sparse Pollen color: RHS 8C.
   Pistil.—Quantity: One Length: 0.40 cm Stigma color: RHS 144C Style color: RHS 145C Style length: 0.3 cm.
Fruit/seed set: No fruit or seed has been produced.
Disease and insect resistance: Resistance and susceptibility is typical of the species.

### COMPARISON WITH PARENTAL AND COMMERCIAL CULTIVARS

‘Bante Pinka07’ differs from the female parent ‘22-1’ in that ‘Bante Pinka07’ has a lavender-pink flower color, while the female parent has a fuchsia flower color. In addition, ‘Bante Pinka07’ flowers 5 to 7 days earlier and has slightly smaller, darker green foliage than the female parent.

‘Bante Pinka07’ differs from the male parent ‘F13-1 in that ‘Bante Pinka07’ has a darker pink flower color than that of the male parent. In addition, ‘Bante Pinka07’ flowers 2 days earlier and has a more compact habit with better basal branching than the male parent.

‘Bante Pinka07’ differs from the commercial variety ‘Bante Rossa’ (U.S. Plant patent Ser. No. 11/406,006) in that ‘Bante Pinka07’ has a more compact plant habit, with better branching, tighter internodes and smaller foliage. ‘Bante Pinka07’ has flower colors ranging from light to medium yellow to pink and medium pink flowers. ‘Bandana Rose’ flowers range from a medium yellow to deeper pink to rose flowers.

I claim:

1. A new and distinct cultivar of lantana plant as shown and described herein.
